(19) United States
(12) Patent Application Publication
(10) Pub. No.: US 2006/0263367 A1
(43) Pub. Date: Nov. 23, 2006

(54) Bispecific antibody devoid of Fc region and method of treatment using same

(76) Inventors: Georg H. Fey, Neunkirchen am Brand (DE); Joerg Bruenke, Nurnberg (DE)

Correspondence Address: Georg H. Fey, Streitbaumweg 8, Neunkirchen am Brand, D 91077, DE

(21) Appl. No.: 11/136,096

(22) Filed: May 23, 2005

(51) Int. Cl. A61K 39/395; C07K 16/30; C07K 16/46

(52) U.S. Cl. 424/155.1; 530/388.8; 530/391.1

(57) ABSTRACT

Bispecific antibody derivatives are disclosed which are comprised of a first region which binds to a first antigen and a second region which binds to a second antigen different from the first antigen. The first and second regions of the bispecific antibody are each stabilized by an additional internal disulfide bridge, and connected by a flexible polypeptide linker. The bispecific antibody is devoid of an Fc portion and is encoded as a single chain-sequence.

### FIELD OF THE INVENTION

This invention relates generally to the field of antibodies useful in treating patients with leukemias and lymphomas.

### BACKGROUND OF THE INVENTION

Relapse of leukemias and lymphomas caused by minimal residual disease (MRD) cells not eradicated by previous chemo- and radiotherapy still remains a major problem after transplantation of hematopoietic stem cells (Handgretinger, R., Klingebiel, T., Lang, P., Gordon, P. & Niethammer, D. (2003) Megadose transplantation of highly purified haploidentical stem cells: current results and future prospects. Pediatr Transplant, 7 Suppl 3, 51-55.). The first months after allogeneic stem cell transplantation offer an advantageous window of time for the elimination of persisting MRD cells. Early reconstituted donor-derived effector cells, such as NK cells can be used to redirect cellular cytotoxicity against leukemic blasts and to increase graft versus leukemia (GvL) effects, without the induction of graft versus host disease (GvHD). Indeed, in two studies with completely T cell depleted grafts, the relapse rates were not clearly increased, despite delayed T cell regeneration. This observation may be ascribed to the rapid reconstitution of NK cells in those patients (Eyrich, M., Lang, P., Lal, S., Bader, P., Handgretinger, R., Klingebiel, T., Niethammer, D. & Schlegel, P. G. (2001) A prospective analysis of the pattern of immune reconstitution in a paediatric cohort following transplantation of positively selected human leucocyte antigen-disparate haematopoietic stem cells from parental donors. Br J Haematol, 114, 422-432.). Antibody-based therapeutics offer the particular advantage of antigen specificity and the recruitment of immune effector cells. A chimeric antibody targeting CD19 was recently shown to mediate specific lysis of primary ALL blasts with donor-derived effector cells obtained from pediatric leukemia patients after transplantation of purified allogeneic stem cells (Lang, P., Barbin, K., Feuchtinger, T., Greil, J., Peipp, M., Zunino, S. J., Pfeiffer, M., Handgretinger, R., Niethammer, D. & Fey, G. H. (2004) A chimeric CD19 antibody mediates cytotoxic activity against leukemic blasts with effectors from pediatric patients transplanted with T cell depleted allografts. Blood.).

However, some limitations restricting the therapeutic efficacy of conventional monoclonal antibodies are known. Penetration of the tumor is limited by the size of the whole antibody (approx. 150 kDa). Interactions of the Fc domain with Fc receptors on non-cytotoxic cells, e.g. platelets or B cells, or non-activating Fc receptors, such as FcγRIIIb on granulocytes, may also reduce their therapeutic effects (Peipp, M. & Valerius, T. (2002) Bispecific antibodies targeting cancer cells. Biochem Soc Trans, 30, 507-511.). Interaction with inhibitory Fc receptor isoforms, such as FcγRIIb on monocytes/macrophages, may further decrease their cytotoxic activity (Clynes, R. A., Towers, T. L., Presta, L. G. & Ravetch, J. V. (2000) Inhibitory Fc receptors modulate in vivo cytoxicity against tumor targets. Nat Med, 6, 443-446.). In addition, the glycosylation pattern of the IgG1 Fc-region at the amino acid Asn297 influences binding to Fc receptors and the induction of ADCC (Shields, R. L., Lai, J., Keck, R., O'Connell, L. Y., Hong, K., Meng, Y. G., Weikert, S. H. & Presta, L. G. (2002) Lack of fucose on human IgG1 N-linked oligosaccharide improves binding to human Fcgamma RIII and antibody-dependent cellular toxicity. J Biol Chem, 277, 26733-26740.). Finally, Fc receptor polymorphisms may critically determine the clinical response to antibody therapy. This effect was demonstrated for the bi-allelic polymorphism of FcγRIIIA (Val 158 vs. Phe 158) in clinical applications of the CD20 antibody Rituximab (Cartron, G., Dacheux, L., Salles, G., Solal-Celigny, P., Bardos, P., Colombat, P. & Watier, H. (2002) Therapeutic activity of humanized anti-CD20 monoclonal antibody and polymorphism in IgG Fc receptor FcgRIIIa gene. Blood, 99, 754-758.; Weng, W. K. & Levy, R. (2003) Two immunoglobulin G fragment C receptor polymorphisms independently predict response to rituximab in patients with follicular lymphoma. J Clin Oncol, 21, 3940-3947.).

By contrast, bispecific antibodies (bsAbs) have the potential to overcome at least some of the limitations associated with conventional antibodies (Peipp, M. & Valerius, T. (2002) Bispecific antibodies targeting cancer cells. Biochem Soc Trans, 30, 507-511.). BsAbs combine two antigen-binding sites, one directed against a tumor-associated antigen, the other against a trigger molecule on effector cells. Thereby, bsAbs very efficiently recruit cytotoxic effector cells, such as NK cells, T-cells, monocytes/macrophages or granulocytes to the tumor cells and mediate elimination of the tumor cells via ADCC or phagocytosis. For the induction of cellular cytotoxicity, activation of effector cells is a critical requirement, which is achieved by antibody binding to cytotoxic trigger molecules, such as CD3 on T cells, CD16 on NK cells, CD64 on activated neutrophils and monocytes/macrophages, or CD89 on neutrophils (Peipp, M. & Valerius, T. (2002) Bispecific antibodies targeting cancer cells. Biochem Soc Trans, 30, 507-511.). Initially, bsAbs were generated by the hybrid-hybridoma technique, but subsequently different types of genetically engineered bispecific antibody-derivatives were designed, e.g. diabodies, mini-antibodies, single chain diabodies, and bispecific single chain Fv antibodies (bsscFvs) (Peipp, M. & Valerius, T. (2002) Bispecific antibodies targeting cancer cells. Biochem Soc Trans, 30, 507-511.). Single-chain diabodies and bsscFvs have the particular advantage of being single-chain polypeptides and, therefore, are easier to produce in a homogeneous and defined final state.

One of the most interesting targets for antibody therapy on malignant human B cells is CD19 (Grossbard, M. L., Press, O. W., Appelbaum, F. R., Bernstein, I. D. & Nadler, L. M. (1992) Monoclonal antibody-based therapies of leukemia and lymphoma. Blood, 80, 863-878.). This surface antigen is expressed on nearly all developmental stages of the B cell lineage. More importantly, CD19 is neither shed from the cell surface, nor lost from tumor cells, nor expressed on hematopoietic stem cells, T cells, or other non-lymphoid cells. Thus, CD19 is a particularly attractive target antigen for antibody therapy. So far, CD19 antibodies have been investigated in various formats for therapeutic studies in cell culture and in vivo (Hekman, A., Honselaar, A., Vuist, W. M., Sein, J. J., Rodenhuis, S., ten Bokkel Huinink, W. W., Somers, R., Rumke, P. & Melief, C. J. (1991) Initial experience with treatment of human B cell lymphoma with anti-CD19 monoclonal antibody. Cancer Immunol Immunother, 32, 364-372.; Pietersz, G. A., Wenjun, L., Sutton, V. R., Burgess, J., McKenzie, I. F., Zola, H. & Trapani, J. A. (1995) In vitro and in vivo antitumor activity of a chimeric anti-CD19 antibody. Cancer Immunol Immunother, 41, 53-60.; Lang, P., Barbin, K., Feuchtinger, T., Greil, J., Peipp, M., Zunino, S. J., Pfeiffer, M., Handgretinger, R., Niethammer, D. & Fey, G. H. (2004) A chimeric CD19 antibody mediates cytotoxic activity against leukemic blasts with effectors from pediatric patients transplanted with T cell depleted allografts. Blood.).

The first CD19-directed conventional bsAbs used CD3 as the trigger molecule for the recruitment of T cells as effectors (Haagen, I. A., van de Griend, R., Clark, M., Geerars, A., Bast, B. & de Gast, B. (1992) Killing of human leukaemia/lymphoma B cells by activated cytotoxic T lymphocytes in the presence of a bispecific monoclonal antibody (alpha CD3/alpha CD19). Clin Exp Immunol, 90, 368-375.; Weiner, G. J. & De Gast, G. C. (1995) Bispecific monoclonal antibody therapy of B-cell malignancy. Leuk Lymphoma, 16, 199-207.; Kipriyanov, S. M., Moldenhauer, G., Strauss, G. & Little, M. (1998) Bispecific CD3×CD19 diabody for T cell-mediated lysis of malignant human B cells. Int J Cancer, 77, 763-772.; Loffler, A., Kufer, P., Lutterbuse, R., Zettl, F., Daniel, P. T., Schwenkenbecher, J. M., Riethmuller, G., Dorken, B. & Bargou, R. C. (2000) A recombinant bispecific single-chain antibody, CD19×CD3, induces rapid and high lymphoma-directed cytotoxicity by unstimulated T lymphocytes. Blood, 95, 2098-2103.). Although cell culture data demonstrated significant lytic activity for these [CD19×CD3] bsAbs, the bsAb-mediated crosslinking of CD3 led to nonspecific T-cell activation, causing bsAb-associated toxicity in vivo (Segal, D. M., Weiner, G. J. & Weiner, L. M. (1999) Bispecific antibodies in cancer therapy. Curr Opin Immunol, 11, 558-562.). A very specific type of a [CD19×CD16]-directed recombinant diabody has been reported to trigger NK cell-mediated tumor cell lysis in vitro and in a specific mouse model (Kipriyanov, S. M., Cochlovius, B., Schafer, H. J., Moldenhauer, G., Bahre, A., Le Gall, F., Knackmuss, S. & Little, M. (2002) Synergistic antitumor effect of bispecific CD19×CD3 and CD19×CD16 diabodies in a preclinical model of non-Hodgkin's lymphoma. J Immunol, 169, 137-144.).

CD16 is the low affinity receptor for IgG (FcγRIII), which is constitutively expressed as a transmembrane isoform on the surface of NK cells and macrophages (CD16a), and as a GPI-anchored molecule on the surface of neutrophils (CD16b) (Ravetch, J. V. & Kinet, J. P. (1991) Fc receptors. Annu Rev Immunol, 9, 457-492.; van de Winkel, J. G. & Anderson, C. L. (1991) Biology of human immunoglobulin G Fc receptors. J Leukoc Biol, 49, 511-524.). For intracellular signaling, CD16a requires association with the FcRγ chain containing the immunoreceptor tyrosine-based activation motif (ITAM), which triggers downstream signaling. Studies with conventionally coupled bsAbs redirecting NK cells via CD16 demonstrated potent cytolysis of cultured malignant cells and in animal models (Garcia de Palazzo, I., Holmes, M., Gercel-Taylor, C. & Weiner, L. M. (1992) Antitumor effects of a bispecific antibody targeting CA19-9 antigen and CD16. Cancer Res, 52, 5713-5719.; Hombach, A., Jung, W., Pohl, C., Renner, C., Sahin, U., Schmits, R., Wolf, J., Kapp, U., Diehl, V. & Pfreundschuh, M. (1993) A CD16/CD30 bispecific monoclonal antibody induces lysis of Hodgkin's cells by unstimulated natural killer cells in vitro and in vivo. Int J Cancer, 55, 830-836.; Kipriyanov, S. M., Cochlovius, B., Schafer, H. J., Moldenhauer, G., Bahre, A., Le Gall, F., Knackmuss, S. & Little, M. (2002) Synergistic antitumor effect of bispecific CD19×CD3 and CD19×CD16 diabodies in a preclinical model of non-Hodgkin's lymphoma. J Immunol, 169, 137-144.). Therefore, clinical trials with CD16-directed bsAbs were initiated (Weiner, L. M., Clark, J. I., Davey, M., Li, W. S., Garcia de Palazzo, I., Ring, D. B. & Alpaugh, R. K. (1995) Phase I trial of 2B1, a bispecific monoclonal antibody targeting c-erbB-2 and FcgRIII. Cancer Res, 55, 4586-4593.; Hartmann, F., Renner, C., Jung, W., Deisting, C., Juwana, M., Eichentopf, B., Kloft, M. & Pfreundschuh, M. (1997) Treatment of refractory Hodgkin's disease with an anti-CD16/CD30 bispecific antibody. Blood, 89, 2042-2047.). However, hybrid-hybridoma antibodies have problems with immunogenicity. Further, such antibodies generate undesired side effects caused by the presence of Fc-domains. Still further, there are difficulties in producing sufficient amounts of clinical-grade material.

### SUMMARY OF THE INVENTION

Bispecific antibodies are disclosed which are produced as a single chain polypeptide. More particularly, the bispecific antibodies of the invention are devoid of an Fc region and are produced as a single chain polypeptide and not the product of two chains separately synthesized and then bound together. The resulting single chain bispecific antibody is a molecule which is almost completely functional and useful. Thus, the molecule can be very efficiently produced in that 90% or more, 95% or more or even as much as 100% of the molecule is functional and useful as a bispecific antibody. The elimination of the Fc portion avoids undesirable effector functions. The antibody can include one or more disulfide bonds bridging the variable light (V_{L}) and variable heavy (V_{H}) chains. The bispecific antibodies of the invention can be formulated into injectable formulations comprised of the bispecific antibody and a carrier.

As aspect of the invention is a bispecific antibody derivative. This antibody derivative includes certain characteristics and features which provide advantages over conventional bispecific antibody derivatives. One advantage is due to the lack of an Fc portion which makes it possible to more efficiently produce the antibody derivative making substantially all of it useful and operable. Thus, an aspect of the invention is not including a particular portion or more specifically not including a Fc portion. Thus, although the bispecific antibody derivative of the invention can be said to “comprise” specific components it is also correct to say that the bispecific antibody derivative of the invention “consists essentially of” or alternatively “consists of” particular components. Those components are a first region which binds to a first antigen, a second region which binds to a second antigen different from the first antigen, a flexible polypeptide linker connecting the first and second regions and a disulfide bridge within each of the first and second regions. The antibody derivative of the invention does not include an Fc portion and preferably does not include any other portions although it does include a single stabilizing disulfide bridge within each of the first and second portions which disulfide bridge within each of these portions results in a half life of the antibody derivative which is 50% or more longer as compared to the same component in the absence of such disulfide bridges.

In order to demonstrate the actual operability of such a bispecific antibody such is described herein a bispecific antibody which binds to the antigen CD19 and CD16 is provided as an example. However, the bispecific antibody, formulations containing such and methods of treatment using such as well as other aspects of the invention are more generally applicable.

A specific embodiment of the invention comprises therapeutic formulations comprised of a recombinant bispecific single-chain Fv antibody (bsscFv), directed against the B-cell antigen CD19 and the low affinity Fc-receptor FcγRIII (CD16), are useful in the treatment of patients with leukemias and lymphomas. The Fc-portions of whole antibodies were deliberately eliminated in this construct to avoid undesired effector functions. A stabilized bsscFv, ds[CD19×CD16], was generated, in which disulfide bonds bridging the respective variable light (V_{L}) and variable heavy (V_{H}) chains were introduced into both component scFvs.

After production in 293T cells and chromatographic purification, ds[CD19×CD16] specifically and simultaneously bound both the CD19 and CD16 antigens. The serum stability of ds[CD19×CD16] was increased more than three-fold when compared to the unstabilized counterpart, while other biological properties were not affected by these mutations.

In antibody-dependent cellular cytotoxicity (ADCC) experiments, ds[CD19×CD16] mediated specific lysis of both CD19-positive malignant human B-lymphoid cell lines and primary tumor cells from patients with B-CLL or B-ALL. NK cells, MNCs from healthy donors, and in some instances MNCs isolated from patients after allogeneic stem cell transplantation were used as effectors. This shows that ds[CD19×CD16] formulated into an injectable formulation is useful in the treatment of CD19⁺ B-lineage malignancies.

A feature of the invention is that the antibody does not contain any Fc-portions.

Another aspect of the invention is that the antibody consists essentially of a single polypeptide chain which polypeptide chain is synthesized as a single chain and is not the product of two chains separately synthesized and separately bound together.

Another aspect of the invention is that due to the method of synthesis of the antibody as a single chain, substantially 100% of the molecule synthesized are functional and useful.

Another aspect of the invention is a bispecific antibody which consists essentially of a single chain polypeptide which polypeptide sequence consists essentially of a sequence which binds to a first epitope and a sequence which binds to a second epitope and which is specifically devoid of other sequences such as an Fc portion.

In yet another aspect of the invention such a bispecific antibody is disclosed wherein the variable light and variable heavy chains are interconnected by one or more disulfide bonds.

In still yet another aspect of the invention such a bispecific antibody is disclosed as present within a carrier as an injectable formulation.

In yet another aspect of the invention a method of treatment is disclosed whereby a formulation comprised of a carrier and a bispecific antibody of the invention is injected into an individual in need of treatment.

These and other aspects, advantages and objects of the invention will become apparent to those skilled in the art upon reading this disclosure.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 includes 1A which is a schematic block diagram of an expression vector for the antibody derivative of the invention, and 1B is an image of a Western Blot showing lanes 1, and 2 representing consecutive elution fractions of the bsscFv after affinity chromatography with Ni NTA agarose beads; and 1C shows purified bsscFv by SDS-PAGE in lanes 1 and 2 which are consecutive elution fractions.

FIG. 2 includes FIG. 2A which shows graphs i, ii, iii and iv of the number of cells versus fluorescent intensity; and FIG. 2B shows four graphs i, ii, iii, and iv of flow cytometry analysis under four different conditions.

FIG. 3 includes FIG. 3A which is a graph of binding activity versus time and FIG. 3B which is a graph of binding activity versus time.

FIGS. 4A and 4B are each graphs of specific lysis in % versus antibody concentration (4A) and versus E/T ratio (4B).

FIG. 5 is a graph of specific lysis in % for four different antibodies and a control.

FIG. 6 is a graph of specific lysis in % for six patient samples with two different antibody constructs and a control for each patient sample.

FIG. 7 includes FIGS. 7A and 7B which are each a graph of specific lysis in % versus the E:T ratio.

### DETAILED DESCRIPTION OF THE INVENTION

Before the present antibodies and methods of using such are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “an antibody” includes a plurality of such antibodies and reference to “the carrier” includes reference to one or more carriers and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

Abbreviations used:

ADCC, antibody-dependent cellular cytotoxicity;

ALL, acute lymphatic leukemia;

bsAb, bispecific antibody;

bsscFv, bispecific single-chain Fv;

CD16ex, extracellular domain of human CD16;

CDC, complement dependent cytotoxicity;

CLL, chronic lymphocytic leukemia;

GFP, green fluorescent protein;

GvHD, graft versus host disease;

GvL, graft versus leukemia;

HAMA, human anti-mouse antibodies;

ITAM, immunoreceptor tyrosine based activation motif;

MNC, mononuclear cells;

MRD, minimal residual disease;

PMN, polymorphonuclear cells;

scFv, single-chain fragment variable.

### INVENTION IN GENERAL

A bispecific antibody derivative is disclosed which is useful in treating human patients which have malignant cells. The antibody derivative is comprised of a first region which binds to CD19 and a second region which binds to CD16. The first and second region each comprise a single-chain fragment variable (scFv) component which components are stabilized with a disulfide bridge. The antibody derivative of the invention does not include an Fc-region and has enhanced serum stability.

Lysis of CD19⁺ MRD cells by donor-derived NK cells, mediated by a chimeric CD19 antibody, was previously reported (Lang et al, 2004). However, this chimeric antibody is still afflicted with the limitations of conventional antibodies. To overcome some of these limitations a recombinant bsscFvs was used instead of a whole antibody to eliminate the Fc-region, which we believed to be one of the causes of the problems in the past. Further, we have found that the Fc-region is not strictly required for the present purpose. The instability of scFvs has been problematic when the antibody was used therapeutically. In order to enhance the serum stability of this recombinant bsscFv, disulfide bridges were introduced to stabilize both scFv components of the antibody derivatives of the invention. The combination of these two advances provide improved results showing that ds[CD19×CD16] triggered potent ADCC of lymphoma cell lines and primary human B-CLL and B-lineage ALL cells.

As can be understood from the above and the examples described in detail below the antibody of the invention is not comprised of any Fc-portions. This is a major difference from conventional bi-specific antibodies. The Fc-portions tend to stick to Fc-receptors on a number of cells that are not targets of the construct. Accordingly, an antibody which does include an Fc-portion can be non-specific. This non-specific sticking is a major undesirable side effect of antibodies. In that the antibody of the invention is not comprised of any Fc-domain it avoids these undesirable side effects. The antibody of the invention consists essentially of or consists only of a single polypeptide chain. However, most antibodies are comprised of two separate polypeptide chains which must be produced in a cell a similar concentrations and thereafter must find each other in order to form a 1:1 complex. The 1:1 complex of such antibodies is the functionally active species. The formation of the 1:1 complex from two component polypeptide chains is a process, which can be disturbed in a number of different ways. When two chains are separately expressed from different promoters they can be synthesized in different concentrations. This would lead to a 1:1 complex formation at a rate which is determined by the second order chemical kinetics by the concentration of the limiting partner. The excess of the other partner is wasted and no complex is formed with the excess partner. In addition, when the chains are synthesized separately they are not identical and therefore can have different distributions profiles, stabilities and half-lives. All of these factors contribute to a final outcome that only a fraction of the two component chains produced actually ever form the 1:1 complex and the remainder are wasted. Thus, the methodology of the present invention which produces the antibody as a single chain is fundamentally different as every polypeptide synthesized is comprised of both components which are linked to one another. Accordingly, in accordance with the present invention substantially 100% of the molecules synthesized can and do form the desired functional antibody. Thus, the method of producing an antibody in accordance with the invention is a more efficient methodology and the resulting antibody is a more efficient design.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

Materials and Methods

Antibodies and Bispecific Antibodies

The hybridoma cell line 3G8 (FcγRIII, CD16; mIgG1) (Fleit, H. B., Wright, S. D. & Unkeless, J. C. (1982) Human neutrophil Fcg receptor distribution and structure. Proc Natl Acad Sci USA, 79, 3275-3279.) was from the American Type Cell Culture Collection (ATCC, Manassas, Va.). The 4G7 hybridoma (CD19, mIgG1) (Meeker et al, 1984) was provided by Dr. R. Levy (Stanford University, Palo Alto, Calif.). The monoclonal antibodies used for detection of recombinant proteins were Penta-His (Qiagen, Hilden, Germany), horseradish peroxidase (HRP)-coupled sheep anti-mouse IgG (Dianova, Hamburg, Germany), phycoerythrin (PE)-coupled goat anti-mouse IgG (DAKO Diagnostica GmbH, Hamburg, Germany) and PE-coupled donkey anti mouse IgG VL+VH (Dianova, Hamburg, Germany).

Culture of Eukaryotic Cells

Chinese hamster ovary (CHO) cells, stably transfected with a human CD16A cDNA expression construct, were provided by Dr. Jan van de Winkel (University Medical Center, Utrecht, The Netherlands). Leukemia-derived SEM cells (t(4;11)-positive ALL), ARH-77 (mature B cell lymphoma; ATCC), and the hybridomas 3G8 and 4G7 were cultured in RPMI 1640-Glutamax-I medium (Invitrogen, Karlsruhe, Germany), containing 10% fetal calf serum (FCS), 100 units/ml penicillin and 100 μg/ml streptomycin (RF10⁺-medium). Human 293T cells (ATCC) were cultured in DMEM-Glutamax-I medium supplemented with 10% FCS, 100 units/ml penicillin and 100 μg/ml streptomycin.

Bacterial Strains and Plasmids

Escherichia coli XL-1-Blue (Stratagene, Amsterdam, The Netherlands) was used for the amplification of plasmids and cloning. The vector pSecTag2HygroC (Invitrogen, Karlsruhe, Germany) was used for expression in eukaryotic cells.

Construction of Recombinant bsscFv [CD19×CD16] and ds[CD19×CD16

To generate the expression vector for the bsscFv [CD19×CD16], the CD19 4G7 scFv was excised from the vector pSecTag2HygroC-CD19 4G7-GFP (Peipp, M., Saul, D., Barbin, K., Bruenke, J., Zunino, S. J., Niederweis, M. & Fey, G. H. (2004) Efficient eukaryotic expression of fluorescent scFv fusion proteins directed against CD antigens for FACS applications. J Immunol Methods, 285, 265-280.) as a SfiI cassette and inserted into the vector pSecTag2HygroC-Strep-CD16 (J. Bruenke, unpublished data) linearized with SfiI, thus generating the vector pSecTag2HygroC-Strep-CD19 4G7×CD16. Disulfide-stabilization of the scFv components in the bsscFv was achieved by the introduction of cysteine residues into conserved framework regions (Reiter, Y., Brinkmann, U., Kreitman, R. J., Jung, S. H., Lee, B. & Pastan, I. (1994) Stabilization of the Fv fragments in recombinant immunotoxins by disulfide bonds engineered into conserved framework regions. Biochemistry, 33, 5451-5459.) in the vector pSecTag2HygroC-Strep-CD19 4G7×CD16 using the Quikchange® Multi Site-Directed Mutagenesis Kit (Stratagene, Cedar Creek, Tex., USA) following manufacturers instructions. Sequences were confirmed by dideoxynucleotide sequencing (Sambrook, J. & Russel, D. W. (2001) Molecular Cloning. A Laboratory Manual, Ed. 3. Cold Spring Harbour Laboratory Press, Cold Spring Harbor, N.Y.) on an Applied Biosystems automated DNA sequencer (ABI Prism 310 Genetic Analyzer; Perkin Elmer; Ueberlingen, Germany).

Expression and Purification of bsscFv and GFP Fusion Proteins

BsscFvs and GFP fusion proteins were expressed in 293T cells. For this purpose, 10 μg of the expression vector were transfected using the calcium phosphate procedure including 5 mM chloroquine (Sambrook, J. & Russel, D. W. (2001) Molecular Cloning. A Laboratory Manual, Ed. 3. Cold Spring Harbour Laboratory Press, Cold Spring Harbor, N.Y.). After 12 h, the transfection medium was replaced by fresh culture medium. Supernatants were collected every day for 5 days and dialyzed at 4° C. against a 2000-fold excess of a buffer containing 50 mM NaH₂PO₄, 300 mM NaCl, 10 mM imidazole, pH 8.0. Purification of the 6×His-tagged bsscFv was achieved by affinity chromatography with nickel-nitrilotriacetic acid (Ni—NTA) agarose beads (Qiagen) and a final dialysis against PBS. Concentrations of the final purified proteins were determined by colorimetric assay using a Bradford Reagent (Sigma, Taufkirchen, Germany).

Expression of the Chimeric CD19 Antibody

The CD19 4G7chim IgG1 antibody was expressed in Sf21 insect cells as previously published (Lang, P., Barbin, K., Feuchtinger, T., Greil, J., Peipp, M., Zunino, S. J., Pfeiffer, M., Handgretinger, R., Niethammer, D. & Fey, G. H. (2004) A chimeric CD19 antibody mediates cytotoxic activity against leukemic blasts with effectors from pediatric patients transplanted with T cell depleted allografts. Blood.).

SDS-PAGE and Western Blot Analysis

Reducing SDS-PAGE was carried out according to standard procedures (Sambrook, J. & Russel, D. W. (2001) Molecular Cloning. A Laboratory Manual, Ed. 3. Cold Spring Harbour Laboratory Press, Cold Spring Harbor, N.Y.). Gels were stained with Coomassie brilliant blue R250. BsscFvs were detected with a Penta-His antibody. Western Blots were developed with secondary antibodies (sheep anti-mouse IgG coupled to horseradish peroxidase; Dianova), and developed using enhanced chemiluminescence reagents (Amersham Pharmacia Biotech, Freiburg, Germany).

Isolation of Mononuclear Cells (MNCs), Neutrophil Effector Cells (PMNs), and CLL Leukemic Cells

After receiving informed consent, 20 ml of peripheral blood was obtained from healthy volunteers, and both mononuclear and neutrophil effector cells were isolated as described (Elsasser, D., Valerius, T., Repp, R., Weiner, G. J., Deo, Y., Kalden, J. R., van de Winkel, J. G., Stevenson, G. T., Glennie, M. J. & Gramatzki, M. (1996) HLA class II as potential target antigen on malignant B cells for therapy with bispecific antibodies in combination with granulocyte colony-stimulating factor. Blood, 87, 3803-3812.). Purity of PMNs and MNCs was assessed by cytospin preparations and exceeded 95%. Viability of cells was >95%, as tested by trypan blue exclusion. Tumor cells from patients with CD5/CD19 positive chronic lymphocytic leukemia (CLL) were isolated from citrate-buffered peripheral blood by centrifugation over Ficoll.

Pediatric Leukemia Patients

Blood samples were obtained from 3 pediatric patients with acute leukemias after transplantation of T cell depleted grafts from unrelated HLA matched allogeneic or haploidentical donors. The myeloablative conditioning regimes were based on total body irradiation. Patients received a median of 12×10⁶ CD34⁺ progenitor cells per kg of body weight. T cells were depleted on average by 5 logs, with less than 25.000 residual CD3⁺ cells in the grafts. No regular posttransplant pharmacological immunosuppression was administered. Ficollized MNCs were used as effector cells for ADCC reactions.

Positive Selection of CD56⁺ Cells

Peripheral mononuclear cells (MNC) were enriched for NK cells by immuno-magnetic separation with CD56⁺ microbeads as previosly described (Lang, P., Pfeiffer, M., Handgretinger, R., Schumm, M., Demirdelen, B., Stanojevic, S., Klingebiel, T., Kohl, U., Kuci, S. & Niethammer, D. (2002) Clinical scale isolation of T cell-depleted CD56⁺0 donor lymphocytes in children. Bone Marrow Transplant, 29, 497-502.).

Flow Cytometry

Immunofluorescence staining was performed as previously published on a FACSCalibur instrument using CellQuest software (Becton Dickinson) (Bruenke, J., Fischer, B., Barbin, K., Schreiter, K., Wachter, Y., Mahr, K., Titgemeyer, F., Niederweis, M., Peipp, M., Zunino, S. J., Repp, R., Valerius, T. & Fey, G. H. (2004) A recombinant bispecific single-chain Fv antibody against HLA class II and FcγRIII (CD16) triggers effective lysis of lymphoma cells. Br J Haematol, 125, 167-179.). For each sample, 1×10⁴ events were collected and analyses of whole cells were performed using appropriate scatter gates to exclude cellular debris and aggregates. Reconstitution of CD3⁺, CD4⁺, CD8⁺, CD19⁺ and CD56⁺ lymphocytes after transplantation was assessed weekly by FACS analysis until T cell recovery began and was subsequently assessed every 3 months.

Determination of Affinity Constants (K_{D}) for Antibodies and scFv Components by Flow Cytometry

Determination of the affinity constants (K_{D}) of the 4G7 antibody and both scFv components of the bsscFv, 4G7 and 3G8, was performed by flow cytometry using published procedures (Benedict, C. A., MacKrell, A. J. & Anderson, W. F. (1997) Determination of the binding affinity of an anti-CD34 single-chain antibody using a novel, flow cytometry based assay. J Immunol Methods, 201, 223-231.).

Experiments for the determination of antibody K_{D} values were repeated 3-4 times and mean values are reported. Values and graphical analyses were generated using GraphPad Prism Software (GraphPad Software Inc., San Diego, Calif., USA).

ADCC and CDC Assays

ADCC assays were performed as described (Elsasser, D., Valerius, T., Repp, R., Weiner, G. J., Deo, Y., Kalden, J. R., van de Winkel, J. G., Stevenson, G. T., Glennie, M. J. & Gramatzki, M. (1996) HLA class II as potential target antigen on malignant B cells for therapy with bispecific antibodies in combination with granulocyte colony-stimulating factor. Blood, 87, 3803-3812.; Lang, P., Barbin, K., Feuchtinger, T., Greil, J., Peipp, M., Zunino, S. J., Pfeiffer, M., Handgretinger, R., Niethammer, D. & Fey, G. H. (2004) A chimeric CD19 antibody mediates cytotoxic activity against leukemic blasts with effectors from pediatric patients transplanted with T cell depleted allografts. Blood.). For analysis of effects induced by Fc-receptor blockade, antibody 3G8 F(ab)₂ (FcγRIII,CD16) was added at a concentration of 10 μg/ml. Relative inhibition was calculated as follows: % inhibition=(% lysis without blocking antibody−% lysis with blocking antibody)/(% lysis without blocking antibody)×100.

Statistical Analyses

Group data are reported as mean±SEM. Differences between groups were analyzed by unpaired (or, when appropriate, paired) Student's t-test.

Experiments reported here were approved by the Ethics Committee of the University of Erlangen-Nuremberg and the University of Tuebingen, in accordance with the Declaration of Helsinki.

Generation, Expression and Purification of the Recombinant bsscFv ds[CD19×CD16 ]

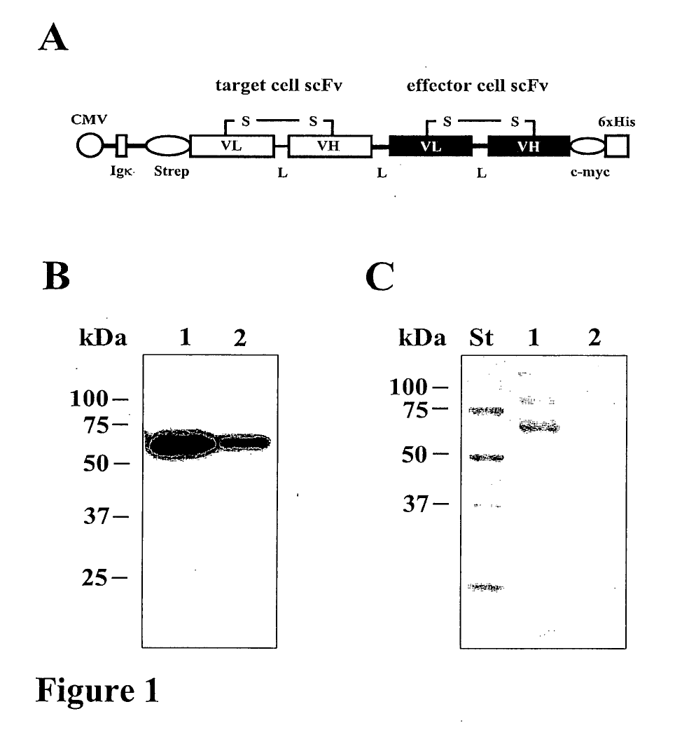

To redirect NK cells against malignant B cells, a recombinant tandem bsscFv was constructed targeting CD19 on malignant B-lymphoid cells and FcγRIII (CD16) on NK cells. To further enhance the stability of this recombinant protein, a stabilized form of this bsscFv ds[CD19×CD16] was generated, in which each scFv component was stabilized by the introduction of a disufide bond bridging the corresponding variable light (VL) and variable heavy (VH) chains (FIG. 1A).

Recombinant bsscFvs were expressed in 293T cells. The secreted bsscFvs were collected from culture supernatants and purified by affinity chromatography with Ni—NTA agarose beads. Protein analysis by SDS-PAGE showed a protein of Mᵣ=58-60 kDa, in close agreement with the calculated molecular mass Mᵣ=59.2 kDa (FIG. 1B). The purified protein was intact, and neither higher molecular weight aggregates nor degradation products were observed. The purity of the enriched protein was estimated from Coomassie stained gels (FIG. 1C). Yields ranged from 1-1.5 mg of purified bsscFv per litre of culture supernatant.

FIG. 1. Design and purification of the recombinant bsscFv ds[CD19×CD16].

FIG. 1A Block-structure of the expression vector for the bsscFv. CMV, cytomegalovirus early promotor; Igκ, secretion leader sequence from the murine Ig kappa light chain; VL, VH, cDNA-segments coding for the immunoglobulin light chain or heavy chain variable regions, respectively; L, cDNA coding for a 20 amino acid flexible linker (Gly₄Ser)₄; Strep, c-myc, 6×His, cDNA coding for a strep, c-Myc or a hexahistidine tag; stabilizing S—S, disulfide bond.

FIG. 1B Western Blot analysis of the bsscFv after affinity chromatography with NiNTA agarose beads. Lanes 1, 2: consecutive elution fractions—revealed with an anti-His-tag antibody.

FIG. 1C Evaluation of the purity and integrity of the purified bsscFv by SDS-PAGE and staining with Coomassie blue: St: size marker; lanes 1, 2: consecutive elution fractions from Ni—NTA agarose beads.

Binding and Stability Characteristics of the Recombinant bsscFv ds[CD19×CD16]

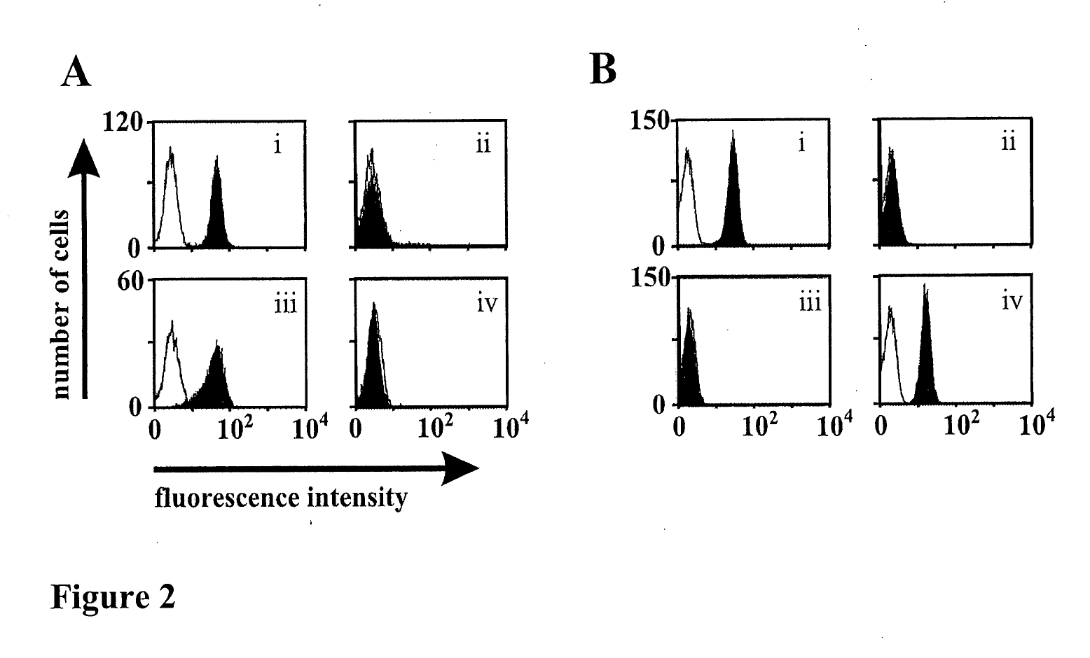

The recombinant bsscFv ds[CD19×CD16] retained the ability to bind to each of the two antigens CD19 and CD16, as evidenced by its ability to specifically bind to the corresponding single-antigen-positive cells (FIG. 2A). To demonstrate simultaneous binding of both scFv components within the same molecule, additional experiments were performed. CD19-positive SEM cells were incubated with the bsscFv and stained with a recombinant fusion protein, consisting of the extracellular domain of CD16 linked to the green fluorescent protein GFP (CD16ex-GFP). In flow cytometry experiments, a fluorescent GFP signal was observed with CD16ex-GFP, but not with a control GFP-fusion protein (FIG. 2B, i).

Thus, both scFv-moieties of one bsscFv-molecule were capable of binding simultaneously to their respective antigens. This result was further supported by competition experiments. Incubation with a molar excess of one of the parental antibodies, directed against CD19 (FIG. 2B, ii) or CD16 (FIG. 2B, iii), resulted in a decrease of the fluorescence signal to baseline levels. Competition with a non-relevant antibody, added in the same molar excess, did not alter the fluorescence signal (FIG. 2B, iv). Therefore, both binding sites of the bsscFv were antigen-specific.

FIG. 2. Specific and simultaneous antigen binding of the bsscFv ds[CD19×CD16].

The four graphs of FIG. 2A show the results of flow cytometry analyses of the bsscFv binding to (i) CD19-positive cells; (ii) CD19-negative cells; (iii) CD16-transfected cells, (iv) untransfected control cells. Black peaks: signals obtained with the bsscFv; white peaks: the signals obtained with a non-relevant scFv.

FIG. 2B is similar to FIG. 2A and shows simultaneous antigen binding of both scFv components contained in the bsscFv. Flow cytometry analyses were performed with the bsscFv on CD19-positive cells. Binding of the bsscFv was revealed by adding a fusion protein consisting of the extracellular domain of CD16 fused to GFP (CD16ex-GFP) (i); black peaks: fluorescent signal produced by CD16ex-GFP; white peaks: fluorescent signal after addition of a non-relevant GFP-fusion protein. The CD16ex-GFP-signal was blocked by addition of a 100-fold molar excess of the parental CD19 and CD16 antibodies 4G7 (ii) and CD16 3G8, respectively (iii); addition of a non-relevant antibody produced no reduction in fluorescence intensity (iv).

To assess, whether stabilization of the scFv components in the bsscFv resulted in an affinity alteration, binding of the scFv components was measured and compared to the unstabilized counterpart. For this purpose, K_{D} was determined as the antibody concentration, at which half maximal binding (half-maximal fluorescence intensity associated with the cells) was reached (Table 1).

TABLE 1

| | K_{D} (M) | K_{D} (M) |
|---|---|---|
| Antibody | CD19 scFv component | CD16 scFv component |
| [CD19 × CD16] | 4.1 × 10⁻⁸ | 6.1 × 10⁻⁸ |
| ds[CD19 × CD16] | 3.8 × 10⁻⁸ | 5.5 × 10⁻⁸ |

^{a} Flow cytometry-based measurement of affinities for both, CD19 and CD16 scFv components in the unstabilized [CD19 × CD16] and disulfide bond stabilized bsscFv ds[CD19 × CD16] calculated by antibody concentrations, at which half maximal binding was observed (n = 3).

The ds[CD19×CD16] displayed affinites in the same nanomolar range as the unstabilized counterpart. Thus, stabilization had not affected the binding affinities to both antigens, CD19 and CD16, respectively. Furthermore, affinities of the scFvs incorporated into the bsscFv were in the range of other published recombinant bispecific antibodies (Kipriyanov, S. M., Moldenhauer, G., Strauss, G. & Little, M. (1998) Bispecific CD3×CD19 diabody for T cell-mediated lysis of malignant human B cells. Int J Cancer, 77, 763-772.; McCall, A. M., Adams, G. P., Amoroso, A. R., Nielsen, U. B., Zhang, L., Horak, E., Simmons, H., Schier, R., Marks, J. D. & Weiner, L. M. (1999) Isolation and characterization of an anti-CD16 single-chain Fv fragment and construction of an anti-HER2/neu/anti-CD16 bispecific scFv that triggers CD16-dependent tumor cytolysis. Mol Immunol, 36, 433445.; Bruenke, J., Fischer, B., Barbin, K., Schreiter, K., Wachter, Y., Mahr, K., Titgemeyer, F., Niederweis, M., Peipp, M., Zunino, S. J., Repp, R., Valerius, T. & Fey, G. H. (2004) A recombinant bispecific single-chain Fv antibody against HLA class II and FcγRIII (CD16) triggers effective lysis of lymphoma cells. Br J Haematol, 125, 167-179.).

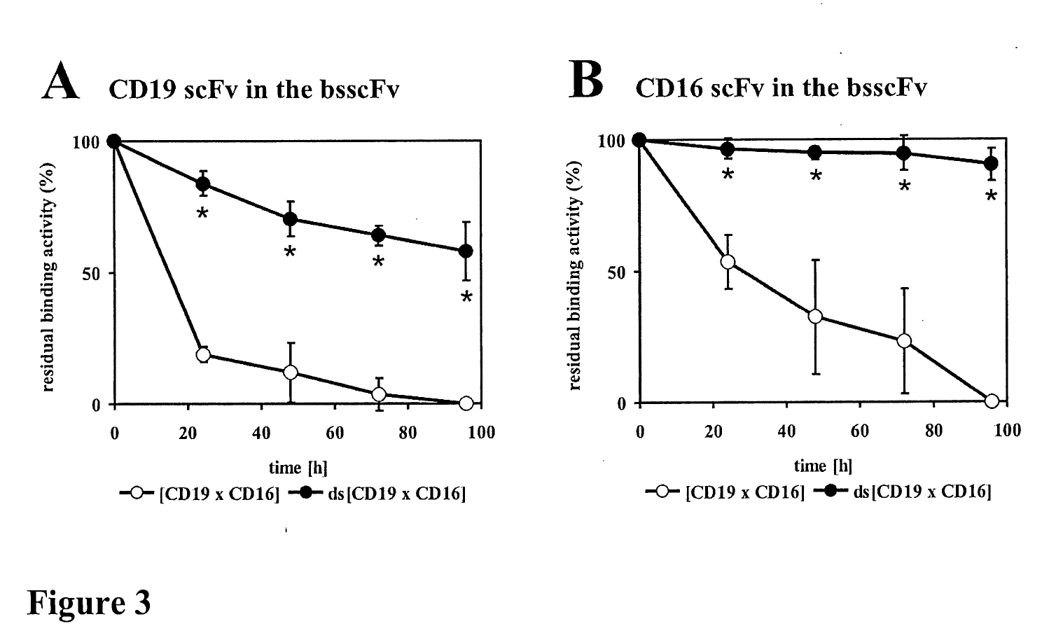

A critical and important factor contributing to the therapeutic usefulness of recombinant antibodies is their stability. Therefore, the plasma stability of the ds[CD19×CD16] was investigated and compared to the unstabilized [CD19×CD16] counterpart (FIG. 3). For this purpose, both bsscFv-constructs were incubated in human serum at 37° C. for prolonged periods of time. At different time points residual binding was quantified by flow cytometry. The half-lifes of the binding sites in the unstabilized bsscFv determined by this method were t_{1/2}=18 h and t_{1/2}=40 h for the CD19 and the CD16 scFv components, respectively. In contrast, the ds[CD19×CD16] retained ≈60% of its antigen binding capacity after 96 h for the CD19 scFv moiety and >90% for the CD16 scFv, respectively. Thus, the introduction of a disulfide bond into each scFv of the bsscFv resulted in a more than three-fold increase of serum stability.

FIG. 3. Serum stability of the disulfide-stabilized bsscFv ds[CD19×CD16] in comparison with the unstabilized bsscFv.

Both, the unstabilized and stabilized [CD1933 CD16] bsscFvs were incubated at subsaturating concentrations of 1 μg/ml in human serum at 37° C. for prolonged periods of time. The residual binding activity was estimated by flow cytometry on antigen-positive cells.

FIG. 3A is a graph showing the stability of the CD19-scFv component in the unstabilized (∘) and the stabilized (●) bsscFv.

FIG. 3B is a graph showing the stability of the CD16-scFv component in the unstabilized (∘) and the stabilized (●) bsscFv. All data are normalized to timepoint t0=100%. Significant values of p<0.05 are indicated by an asterisk (*) Data are presented as mean percentage of residual binding±SEM of 5 independent experiments.

The Recombinant bsscFv ds[CD19×CD16] Mediates Effector Cell Lysis (ADCC)

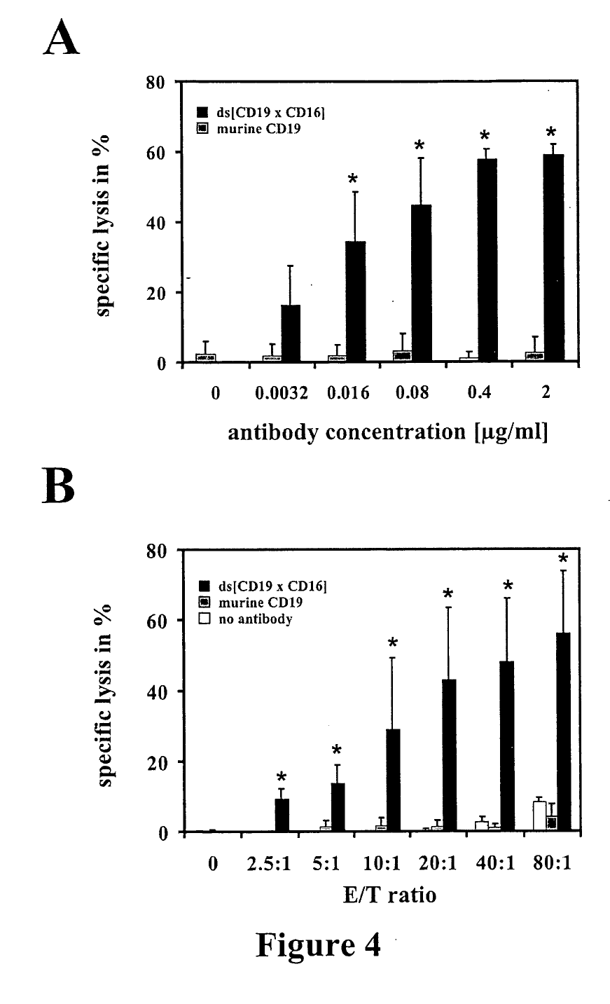

For functional studies, the CD19-positive mature B-cell lymphoma line ARH-77 was used as a target in 3 hour ⁵¹Cr release assays with freshly isolated, unstimulated MNCs as effectors. The ds[CD19×CD16] triggered specific lysis of target cells in a dose-dependent manner with optimal concentrations in the range of 0.4-2 μg/ml (FIG. 4A), while the parental murine CD19 antibody was unable to induce ADCC. The bsscFv displayed effector cell-mediated cytotoxicity against target cells over a broad range of effector-to-target cell ratios down to ratios of total MNCs to target cells of 2.5:1. Target cell lysis in the presence of the parental murine CD19 antibody was not observed. ADCC of target cells by MNCs in the absence of any added antibody construct was only observed at high E/T ratios and specific lysis was always <10% (FIG. 4B). Thus, antibody binding to CD19 alone is not suffient to induce target cell lysis.

FIG. 4. ADCC against CD19-positive tumor cells: role of concentration and E/T ratio.

ADCC against ARH-77 target cells mediated by the bsscFv and freshly isolated human peripheral MNCs as effector cells.

FIG. 4A is a graph showing a constant E/T ratio of 40:1, bsscFv-mediated lysis was concentration-dependent and saturable (black bars). The parental murine CD19 antibody used as a control was unable to induce ADCC (grey bars).

FIG. 4B is a graph showing results in the presence of constant amounts of 0.4 μg/ml of ds[CD19×CD16] (black bars), the extent of specific lysis increased with the E/T ratio in an expected, saturable fashion. Effector cells refers to total number of MNCs, of which only about 10% were NK cells. White bars: without antibody; grey bars: parental murine CD19 antibody. Significant values of p<0.05 are indicated by an asterisk (*). Data are presented as mean percentage lysis±SEM observed with isolated MNCs from at least 3 different donors.

To identify the effector population responsible for this cytotoxicity, peripheral blood from healthy donors was fractionated into MNCs and PMNs and tested in ADCC reactions. Only the MNC fraction, containing the NK cells, demonstrated significant lysis of ARH-77 cells in the presence of the ds[CD19×CD16], whereas PMNs were inactive. As expected by the design, no complement-dependent cytotoxicity (CDC) was observed when using the plasma fraction. Thus, the cell population mediating ADCC was enriched in the MNC fraction, suggesting that CD16a-positive NK cells were the effectors.

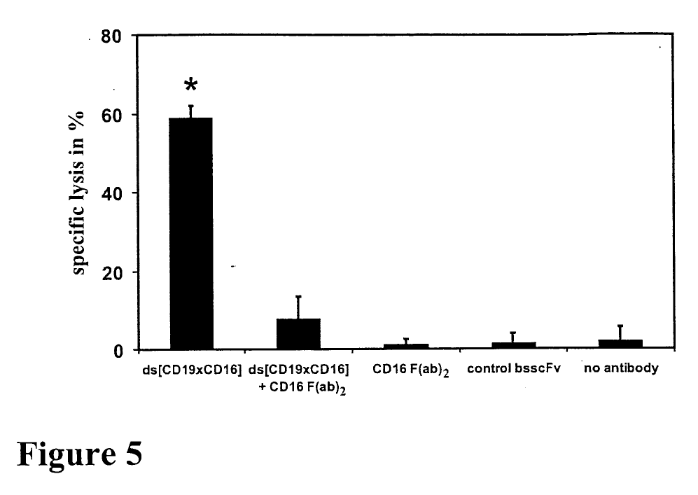

To further confirm that lysis was dependent on CD16, blocking experiments were performed. In the presence of ds[CD19×CD16], MNC-mediated lysis of ARH-77 cells was significantly inhibited by addition of F(ab)₂ fragments of FcΔRIII antibody 3G8 (83.9%±6.6%, FIG. 5). Furthermore, neither the FcγRIII-directed F(ab)₂ fragment nor a recombinant control bsscFv that also binds to CD16, but not to ARH-77 cells, triggered MNC-mediated lysis of target cells (FIG. 5). Thus, non-specific activation of NK cells by ds[CD19×CD16] was not observed under our experimental conditions.

FIG. 5. ADCC of ARH-77 cells is FcγRIII dependent.

The graph of FIG. 5 shows results with bsscFv ds[CD19×CD16] mediated MNC-mediated lysis of ARH-77 target cells at concentrations of 2 μg/ml. Target cell lysis was significantly inhibited (83.9%±6.6%) by addition of F(ab)₂ fragments of FcγRIII antibody 3G8 at concentrations of 10 μg/ml. Neither the 3G8 F(ab)₂ fragments nor a CD16-directed control bsscFv at concentrations of 2 μg/ml were able to induce MNC-mediated lysis. MNCs as effectors also failed to trigger ADCC in the absence of antibody. Significant values of p<0.05 are indicated by an asterisk (*). Data are presented as mean percentage lysis±SEM observed with isolated MNCs from at least 4 different donors.

Cytotoxic Activity of the ds[CD19×CD16] and a Chimeric CD19 Antibody Against Primary CD19-Positive B-CLL Cells

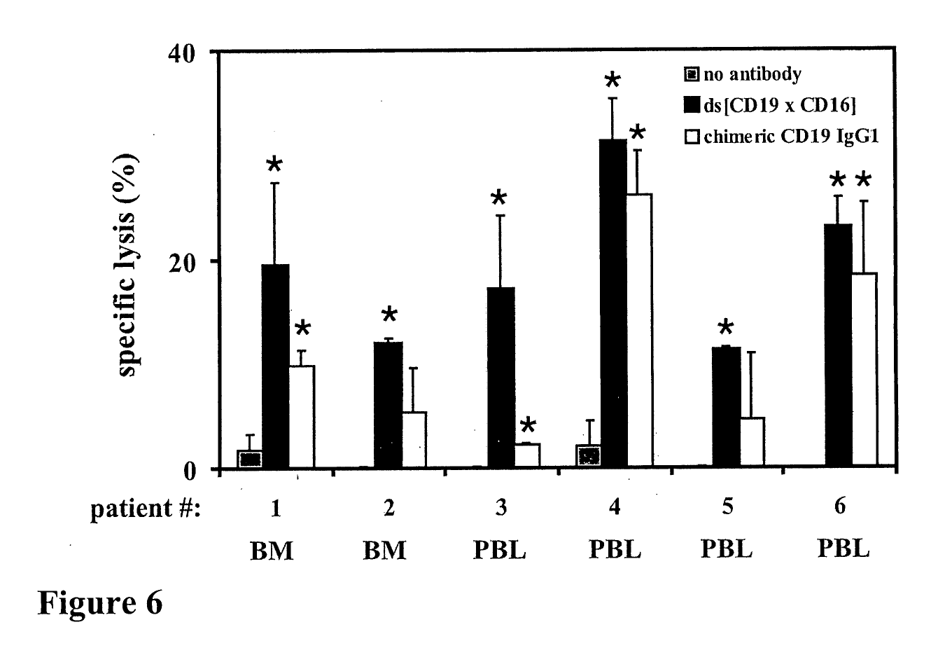

Primary tumor cells are generally more difficult to lyse than lymphoma cell lines. Therefore, the ds[CD19×CD16] was tested in ADCC reactions against freshly isolated and cryopreserved CD19-positive B-CLL cells, derived from bone marrow or peripheral blood, and compared to the corresponding chimeric IgG1 CD19 antibody. Significant lysis of patient samples (p<0.05) was observed in the presence of either the recombinant bispecific in all 6 cases or the chimeric CD19 4G7chim antibody (4/6 cases), whereas effector cell mediated lysis without antibody was consistently low (FIG. 6).

FIG. 6. Lysis of primary B-CLL cells by bsscFv ds[CD19×CD16].

FIG. 6 is a graph showing results with CD5/CD19-positive human B-CLL cells were isolated from bone marrow (BM) or peripheral blood lymphocytes (PBL) of 6 different patients. Antibody-dependent lysis (p<0.05 indicated by *) of B-CLL cells was evaluated in ADCC reactions using the recombinant bsscFv ds[CD19×CD16] at concentrations of 0.4 μg/ml (black bars) or the corresponding chimeric CD19 antibody (CD19 4G7chim) at concentrations of 1.5 μg/ml (open bars). Assays were performed with MNC effector cells from 2 different healthy donors at an E/T ratio of 40:1. The ds[CD19×CD16] mediated lysis of the leukemic cells from all 6 patients, while the chimeric CD19 antibody induced ADCC in 4/6 patient samples. Data are presented as mean percentage lysis±SEM. No attempt was made to search or avoid match of MHC class I between effector and target cells.

Cytotoxic Activity of the ds[CD19×CD16] and Chimeric CD19 Antibody Against Primary CD19-Positive ALL Blasts

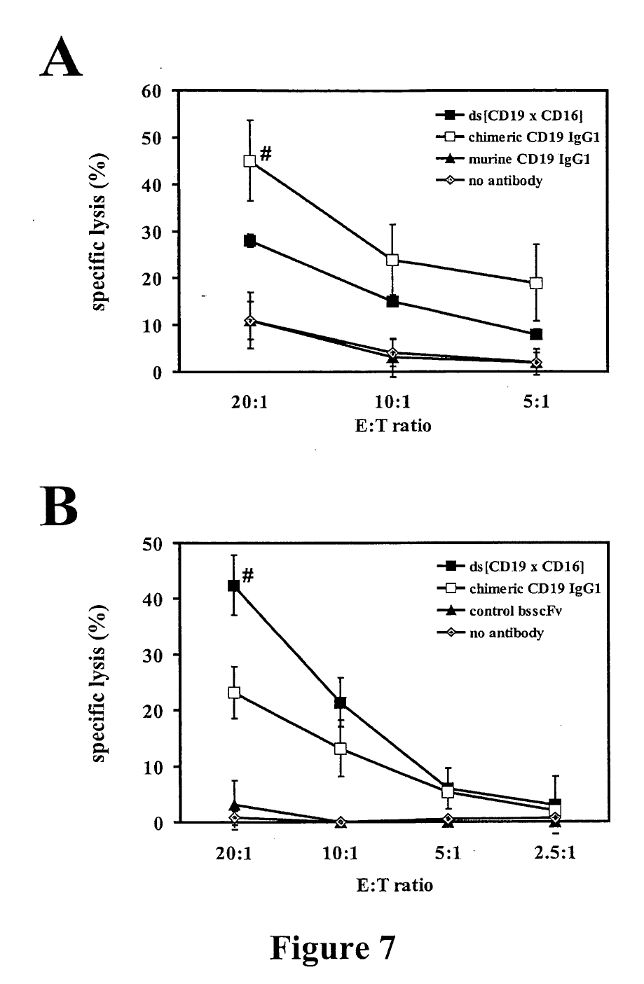

Furthermore, the ds[CD19×CD16] produced specific lysis of primary cryopreserved CD19-positive ALL-blasts from pediatric patients in ADCC reactions mediated by enriched NK cells from unrelated healthy donors at different E/T ratios (FIG. 7A). Although the donor/target pairs produced different levels of spontaneous lysis, NK cells from each donor mediated significantly enhanced lysis (p<0.05) in the presence of both, bispecific ds[CD19×CD16] or chimeric CD19 antibody. Under these experimental conditions at an E/T ratio of 20:1, specific lysis mediated by the ds[CD19×CD16] was somewhat less efficient than by the chimeric IgG1. Lytic activity <10% of NK cells alone was only observed at an E/T ratio of 20:1. As expected, NK cell mediated cytotoxicity was not significantly enhanced in the presence of the murine 4G7 IgG1 hybridoma antibody, used as a control. Stimulation of NK cells with IL-2 at 40 units/ml further increased specific lysis mediated by ds[CD19×CD16] or chimeric CD19 antibody (data not shown). Thus, both CD19-directed antibody constructs, ds[CD19×CD16] and chimeric CD19 antibody, mediated enhanced ADCC against patient ALL blasts in the presence of NK cells from different unrelated donors.

The Recombinant bsscFv ds[CD19×CD16] Mediates Specific Lysis of Primary Leukemic Blasts in the Presence of Effector Cells from Transplanted Patients

To address the question, whether donor-derived effector cells were capable of lysing primary leukemic blasts in conjunction with our bispecific antibody, the ADCC-inducing potential of ds[CD19×CD16] was investigated in ADCC reactions and compared to the chimeric CD19 antibody. For this purpose, MNCs from 3 different patients were isolated after CD34⁺ stem cell transplantation and tested against cryopreserved ALL blasts at different E/T ratios (FIG. 7B) in the presence of either the recombinant bsscFv ds[CD19×CD16] or chimeric CD19 antibody. Although, the donor/target pairs produced different levels of effector cell-mediated lysis, both, ds[CD19×CD16] and CD19 4G7chim, triggered significantly enhanced (p<0.05) lysis of leukemic blasts, while MNCs alone or a CD16-directed control bsscFv did not trigger cellular lysis. In this experimental setting at an E/T ratio of 20:1, killing obtained with the bsscFv was significantly more effective than with CD19 4G7chim (p<0.05). The lytic activity was ascribed to NK cells, because the extent of ADCC obtained with MNCs was proportional to the fraction of CD56⁺/CD16⁺ cells in this mixture (data not shown). Thus, donor-derived effector cells from patients after transplantation were capable of mediating high lytic activity against leukemic blasts in the presence of ds[CD19×CD16].

FIG. 7: The recombinant ds[CD19×CD16] mediates effector cell lysis of primary B-ALL blasts.

FIG. 7A is a graph of results with the recombinant ds[CD19×CD16] induced ADCC of cryopreserved B-ALL blasts by enriched NK cells from different healthy donors at different E/T ratios. Lysis of target cells (<10%) by NK cells alone was only observed at an E/T ratio of 20:1. Significantly enhanced specific lysis (p<0.05) was observed in the presence of the recombinant bsscFv ds[CD19×CD16] at concentrations of 0.4 μg/ml or the chimeric CD19 4G7chim at saturating concentrations of 0.15 μg/ml. Killing mediated by the ds[CD19×CD16] was less efficient than by the chimeric 4G7 under these experimental conditions, while the murine CD19 hybridoma antibody 4G7 triggered no ADCC. Data are presented as mean percentage lysis±SEM observed with enriched NK cells from 3 donors.

FIG. 7B is a graph of results with MNCs isolated from 3 different patients after CD34⁺ stem cell transplantation mediated significant specific lysis (p<0.05) of cryopreserved B-ALL blasts in the presence of ds[CD19×CD16] or CD19 4G7chim. ADCC at different E/T ratios using the bsscFv ds[CD19×CD16] at 0.4 μg/ml, or the chimeric CD19 4G7chim at saturating 0.15 μg/ml. Lysis mediated by the ds[CD19×CD16] was consistently stronger than lysis by the chimeric antibody CD19 4G7chim. No specific lysis was observed when a CD16-directed control bsscFv or MNCs alone were used. Significant differences (p<0.05) between ds[CD19×CD16] and 4G7chim are indicated by (#). Data are presented as mean percentage lysis±SEM observed with isolated MNCs from 3 different patients after transplantation.

An aspect of the invention is a recombinant bispecific scFv molecule in the tandem format which provides an increased serum stability after disulfide-stabilization of both of its scFv components.

Another aspect of the invention is the disulfide-stabilized bsscFv provides greater efficiency in mediating specific lysis of primary ALL-blasts by donor-derived MNCs after transplantation than the chimeric IgG1 antibody CD19 4G7chim.

A disadvantage of scFv and bsscFv-proteins for clinical applications is their instability or relatively short half lives in human serum. In general bsscFvs will have a serum stability of only a few hours to a few days, as demonstrated also by the non-stabilized controls in this study (FIG. 3). To overcome this limitation, disulfide-stabilized Fvs have been generated. It had previously been shown, that disulfide-stabilization significantly increased the serum stability of individual scFv-molecules (Brinkmann, U., Reiter, Y., Jung, S. H., Lee, B. & Pastan, I. (1993) A recombinant immunotoxin containing a disulfide-stabilized Fv fragment. Proc Natl Acad Sci USA, 90, 7538-7542.). However, the present invention shows the simultaneous stabilization of two different scFv components in a tandem format bsscFv increases the serum stability of the entire molecule.

In ADCC experiments with malignant cells obtained either from the bone marrow (BM) or peripheral blood (PBL) of six different adult B-CLL patients, MNCs from unrelated healthy human donors were used as effectors (FIG. 6). Although no deliberate effort was made to assure a mismatch of their MHC class I molecules, lysis was significantly enhanced by addition of the bsscFv molecule in all six cases. Furthermore, the lytic effect was constantly higher with the bsscFv molecule than with the chimeric CD19 antibody, regardless whether the malignant cells were taken from the bone marrow or peripheral blood (FIG. 6). In 4/6 cases (patients 1,2,3,5) the cytotoxic effects obtained with the bsscFv were more than twice as large as those obtained with the chimeric CD19 antibody. It is presently unknown, whether this advantage will also hold true in human patients, but we anticipate certain advantages over the chimeric CD19 antibody, including improved tissue penetration due to its smaller size.

Normally, lytic ability of NK cells is impaired by high expression levels or expression of matched MHC class I molecules on target cells. Apparently, this inhibitory effect, which is due to killer inhibitory receptors (KIR) on the surface of NK cells is overcome in ADCC situations. Here, the lysis-promoting effect produced by the therapeutic antibody apparently compensates the killer inhibitory effects (Lang, P., Barbin, K., Feuchtinger, T., Greil, J., Peipp, M., Zunino, S. J., Pfeiffer, M., Handgretinger, R., Niethammer, D. & Fey, G. H. (2004) A chimeric CD19 antibody mediates cytotoxic activity against leukemic blasts with effectors from pediatric patients transplanted with T cell depleted allografts. Blood 103, 3982-3985.). Rather large variations in the extent of lysis observed for individual pairs of tumor and effector cells (FIG. 6) may be in part due to the variable extent of MHC class I mismatch between the target and effector cells. Other potential causes for this observation may include variations in the expression levels of activating NK cell receptors, such as NKG2D, DAP10 or DAP12.

The ADCC experiments with primary leukemic ALL blasts from pediatric patients with the bsscFv in comparison to the chimeric antibody produced an unexpected observation. When unstimulated enriched NK cells from healthy unrelated donors were used as effectors (FIG. 7A), the chimeric CD19 antibody produced somewhat higher lysis than the bsscFv. However, when unstimulated donor-derived MNCs from transplanted patients were used, then the bsscFv produced a greater extent of lysis (FIG. 7B). In this situation MNCs were used and no attempt was made to enrich NK cells, because the amount of blood cells available from transplanted pediatric patients was too limited to permit enrichment of NK cells. An explanation for this observation might be, that transplanted patients regularly received standard human IgG infusions (200 mg/kg every three weeks) in order to prevent infectious complications. Such infusions are likely to block human Fc receptors as a side effect, and therefore may reduce free binding sites for the chimeric antibody on the effector cells. By contrast, it is conceivable, that the function of the bsscFv is not inhibited under these conditions, because the CD16-specific reading head binds FcγRIII at an epitope outside its Fc-binding site. This experimental setting using donor-derived MNCs comes closest to the in vivo situation, for which our construct was primarily designed, namely the treatment of minimal residual disease (MRD) cells in a post-transplantation setting. In this situation, the bsscFv format had superior properties over the chimeric antibody, which remain to be confirmed by clinical investigations.

CD19 has long been recognized as a potentially very useful target antigen for the therapy of B-lymphoid malignancies, due to its exquisite restriction to the B cell lineage. It is expressed on most B-lineage ALLs, including infant pro-B ALLs, which usually lack CD20, and therefore appears to be particularly attractive for the treatment of CD20-negative pediatric leukemias. Consequently, CD19-directed antibodies have been investigated for therapeutic use against human B-lymphoid malignancies, but until now, therapeutic IgG antibodies have not produced clinical benefits comparable to those of CD20 antibodies (Hekman, A., Honselaar, A., Vuist, W. M., Sein, J. J., Rodenhuis, S., ten Bokkel Huinink, W. W., Somers, R., Rumke, P. & Melief, C. J. (1991) Initial experience with treatment of human B cell lymphoma with anti-CD19 monoclonal antibody. Cancer Immunol Immunother, 32, 364-372.). In addition, conventional bsAbs targeting CD19 were generated for the recruitment of T cells via CD3. These bsAbs were effective in vitro (Bohlen, H., Hopff, T., Manzke, O., Engert, A., Kube, D., Wickramanayake, P. D., Diehl, V. & Tesch, H. (1993a) Lysis of malignant B cells from patients with B-chronic lymphocytic leukemia by autologous T cells activated with CD3×CD19 bispecific antibodies in combination with bivalent CD28 antibodies. Blood, 82, 1803-1812.; Bohlen, H., Manzke, O., Patel, B., Moldenhauer, G., Dorken, B., von Fliedner, V., Diehl, V. & Tesch, H. (1993b) Cytolysis of leukemic B-cells by T-cells activated via two bispecific antibodies. Cancer Res, 53, 4310-4314.; Haagen, I. A., Geerars, A. J., de Lau, W. B., Clark, M. R., van de Griend, R. J., Bast, B. J. & de Gast, B. C. (1994) Killing of autologous B-lineage malignancy using CD3×CD19 bispecific monoclonal antibody in end stage leukemia and lymphoma. Blood, 84, 556-563.; Csoka, M., Strauss, G., Debatin, K. M. & Moldenhauer, G. (1996) Activation of T cell cytotoxicity against autologous common acute lymphoblastic leukemia (cALL) blasts by CD3×CD19 bispecific antibody. Leukemia, 10, 1765-1772.) and in animal models (Demanet, C., Brissinck, J., Moser, M., Leo, O. & Thielemans, K. (1992) Bispecific antibody therapy of two murine B-cell lymphomas. Int J Cancer Suppl, 7, 67-68.; Bohlen, H., Manzke, O., Titzer, S., Lorenzen, J., Kube, D., Engert, A., Abken, H., Wolf, J., Diehl, V. & Tesch, H. (1997) Prevention of Epstein-Barr virus-induced human B-cell lymphoma in severe combined immunodeficient mice treated with CD3×CD19 bispecific antibodies, CD28 monospecific antibodies, and autologous T cells. Cancer Res, 57, 1704-1709.; Daniel, P. T., Kroidl, A., Kopp, J., Sturm, I., Moldenhauer, G., Dorken, B. & Pezzutto, A. (1998) Immunotherapy of B-cell lymphoma with CD3×19 bispecific antibodies: costimulation via CD28 prevents “veto” apoptosis of antibody-targeted cytotoxic T cells. Blood, 92, 4750-4757.), but not in first clinical trials (De Gast, G. C., Van Houten, A. A., Haagen, I. A., Klein, S., De Weger, R. A., Van Dijk, A., Phillips, J., Clark, M. & Bast, B. J. (1995) Clinical experience with CD3×CD19 bispecific antibodies in patients with B cell malignancies. J Hematother, 4, 433-437.; Haagen, I. A. (1995) Performance of CD3×CD19 bispecific monoclonal antibodies in B cell malignancy. Leuk Lymphoma, 19, 381-393.).

More recently, recombinant bsscFvs comprising only antibody variable domains have been constructed. These molecules are expected to be less immunogenic than complete antibodies and can be produced at relatively high yields in a more defined final state. The currently most advanced recombinant protein in this format is a [CD19×CD3] bsscFv (Loffler, A., Kufer, P., Lutterbuse, R., Zettl, F., Daniel, P. T., Schwenkenbecher, J. M., Riethmuller, G., Dorken, B. & Bargou, R. C. (2000) A recombinant bispecific single-chain antibody, CD19×CD3, induces rapid and high lymphoma-directed cytotoxicity by unstimulated T lymphocytes. Blood, 95, 2098-2103.). For the particular purpose of our work, the elimination of MRD cells in pediatric ALL patients after transplantation of T cell depleted grafts, T cells clearly are not the ideal class of effector cells, because of their delayed reconstitution (Eyrich, M., Lang, P., Lal, S., Bader, P., Handgretinger, R., Klingebiel, T., Niethammer, D. & Schlegel, P. G. (2001) A prospective analysis of the pattern of immune reconstitution in a paediatric cohort following transplantation of positively selected human leucocyte antigen-disparate haematopoietic stem cells from parental donors. Br J Haematol, 114, 422432.). NK cells and granulocytes show much faster reconstitution, and, therefore, CD16 was the more promising choice of a trigger molecule on the available population of effector cells: the NK cells. It is also important to note, that in the first few months after transplantation the MRD cells are usually few, and high effector-to-target cell ratios can be achieved.

CD16 has been appreciated by other authors as a potent trigger molecule on the surface of NK cells (Gessner, J. E., Heiken, H., Tamm, A. & Schmidt, R. E. (1998) The IgG Fc receptor family. Ann Hematol, 76, 231-248.). CD16 antibodies, such as 3G8 used in this study, bind FcγRIII outside of the Fc-binding pocket, activate NK cells, and induce cytotoxic responses. Some properties of CD16 may also limit its use as a trigger molecule. Among these is the inability of CD16 antibodies to discriminate between the CD16a isoform present on NK cells and macrophages, which is capable of triggering a cytolytic response, and the GPI-linked CD16b isoform present on neutrophilic granulocytes, which is unable to mediate ADCC. In addition, soluble CD16 is present in considerable concentrations in human plasma (Koene, H. R., de Haas, M., Kleijer, M., Roos, D. & von dem Borne, A. E. (1996) NA-phenotype-dependent differences in neutrophil Fcg RIlIb expression cause differences in plasma levels of soluble Fcg RIII. Br J Haematol, 93, 235-241.) and may compete the interaction with FcγRIII on the surface of NK cells. However, CD16-directed bsAbs have been successfully used in clinical trials, although none have advanced past stage II and none have yet been approved for clinical application. Interestingly, the cytotoxicity of these bsAbs was not inhibited by the presence of CD16-positive PMNs in ADCC assays, a still unexplained observation (Weiner, L. M., Alpaugh, R. K., Amoroso, A. R., Adams, G. P., Ring, D. B. & Barth, M. W. (1996) Human neutrophil interactions of a bispecific monoclonal antibody targeting tumor and human Fc gamma RIII. Cancer Immunol Immunother, 42, 141-150.). In addition, the in vivo cytotoxicity of CD16-directed bsAbs was not compromised by competition with CD16b on neutrophils. This effect was also observed in preclinical studies and phase I/II clinical trials of patients with refractory Hodgkin's disease treated with a [CD30×CD16] bsAb (Hartmann, F., Renner, C., Jung, W., da Costa, L., Tembrink, S., Held, G., Sek, A., Konig, J., Bauer, S., Kloft, M. & Pfreundschuh, M. (2001) Anti-CD16/CD30 bispecific antibody treatment for Hodgkin's disease: role of infusion schedule and costimulation with cytokines. Clin Cancer Res, 7, 1873-1881.). These reported properties of CD16-directed bsAbs provided the basis for our current study and the anticipation, that recombinant bsscFv constructs directed against CD16 as the trigger molecule may be therapeutically useful. Based on the results of this study we conclude, that the format of the tandem bsscFv may have distinct advantages over conventional bsAbs used so far by other authors. In fact, this particular format allows investigators to fully exploit the perceived benefits of CD19 as a target molecule, which had remained below expectations when other formats of CD19-directed antibody-derived therapeutics were used. The data presented here provide a clear impetus for further in vivo evaluation of [CD19×CD16] bsscFvs.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

That which is claimed is:

1. A bispecific antibody derivative, comprising:
a first region which binds to a first antigen;
a second region which binds to a second antigen different from the first antigen;
a flexible polypeptide linker connecting the first and second regions, and
a disulfide bridge within each of the first and second regions;
wherein the bispecific antibody is devoid of an Fc portion.

2. The antibody derivative of claim 1, wherein the antibody derivative is a bispecific single chain Fv antibody (bsscFv).

3. The antibody derivative of claim 1, characterized by a half-life 50% or more longer than a half-life of the antibody derivative in the absence of a disulfide bridge.

4. The antibody derivative of claim 1, wherein the antibody derivative is a single chain polypeptide.

5. The antibody derivative of claim 1, wherein each scFv component is stabilized on a tandem format.

6. The antibody derivative of claim 1, wherein the derivative consists essentially of the first region, the second region, the flexible polypeptide linker connecting the first and second regions and at least one stabilizing disulfide bridge within each of the first and second regions.

7. The bispecific antibody derivative of claim 1, wherein the antibody consists only of the first region, the second region, flexible polypeptide linker connecting the first and second regions and a flexible polypeptide linker connecting the first and second regions.

8. A bispecific antibody derivative, comprising:
a first region which binds B-cell antigen CD19 on malignant human cells; and
a second region which binds CD16, the human Fc-receptor FcγRIII;
wherein the first region and the second region each comprise a single-chain fragment variable (scFv) component stabilized by a disulfide bridge and further wherein the bispecific antibody derivative is devoid of an Fc-portion.

9. The antibody derivative of claim 8, wherein the antibody derivative is a bispecific single chain Fv antibody (bsscFv).

10. The antibody derivative of claim 8, characterized by a half-life 50% or more longer than a half-life of the antibody derivative in the absence of a disulfide bridge.

11. The antibody derivative of claim 8, wherein the antibody derivative is a single chain polypeptide.

12. The antibody derivative of claim 8, wherein each scFv component is stabilized on a tandem format.

13. A method of treatment, comprising:
administering to a patient a formulation comprising:
a pharmaceutically acceptable carrier; and
a bispecific antibody derivative comprising a first region which binds to a first antigen and a second region which binds to a second antigen different from the first antigen, and a flexible polypeptide linker connecting the two regions, wherein the bispecific antibody derivative is devoid of an Fc-portion and is encoded as a single-chain fragment variable component.

14. A method of treatment, comprising:
administering to a patient a formulation comprising:
a pharmaceutically acceptable carrier; and
a bispecific antibody comprising a first region which binds CD19 on malignant human cells, a second region which binds CD16 of a human Fc-receptor FcγRIII, wherein the first region and the second region are each comprised of a single-chain fragment variable (scFv) component which components are each stabilized by a disulfide bridge and further wherein the bispecific antibody derivative is devoid of an Fc-portion.

15. A method of treatment, comprising the steps of:
(a) subjecting a patient to a treatment protocol chosen from chemotherapy and radiotherapy;
(b) administering stem cells to the patient after the treatment protocol (a); and
(c) administering to the patient a formulation comprising:
a pharmaceutically acceptable carrier; and
a bispecific antibody comprising a first region which binds CD19 on malignant human cells, a second region which binds CD16 of a human Fc-receptor FcγRIII, wherein the first region and the second region are each comprised of a single-chain fragment variable (scFv) component which components are each stabilized by a disulfide bridge and further wherein the bispecific antibody derivative is devoid of an Fc-portion.

16. The method of claim 15, wherein the patient suffers from a CD19⁺ lineage malignancy.

17. A method of producing a bispecific antibody derivative, comprising:
synthesizing a single polypeptide chain comprising a first region which binds B-cell antigen CD19 on malignant human cells and a second region which binds CD16 of a human Fc-receptor FcγRIII.

18. The method as claimed in claim 17, further comprising:
creating a disulfide-stabilization bond between a cysteine residue in the V_{H} and V_{L}-portion of each binding region.

19. The method as claimed in claim 17, wherein the single polypeptide chain is synthesized from a sequence encoding the single chain which sequence encoding the single chain is connected to a single promoter.
